SEQUENCE LISTING (2 sequences)

SEQ ID NO: 1 - DNA, 657 bp, synthetic construct
  source 1..657: mol_type = other DNA
atgtcccttcgcattaacgatatcgccccggacttcaccgctgaaacgacgcagggcgaaattaagtttcatgattggattggggacggatgggctattctgtttagtcacccaaaaaatttcacgcccgtatgcaccactgaactgggtacgatggctgggttagaaggtgaatttaagaaaagaaatgtgaaaattattggcatatctgtagaccctgttgcttcacatgacaaatggcaggcagatatcaagacagccactggacactcagtgcactatccacttatcggtgataaagacctgaaggtcgccaaactgtatgatatgttaccagccggtgcaggcgaaacgtcagaaggtcgcaccccagcggataatgctacggtgcgtagtgtgtatgtaattggtccggataaaaaaattaaactggttctgacctaccctatgacgactggccgcaattttgatgaaattttaagagctgttgattcgatgcaactgaccgctaaacaccaagttgctacgccggcaaactggaaacaaggagaagatgttataatcaccgcagccgtctcaaatgaagatgcaataaaacggtttggggcgtatgaaaccatccttccgtacctgagaaaaactaaacaacctagcgcg

SEQ ID NO: 2 - protein, 219 aa, synthetic construct
MSLRINDIAPDFTAETTQGEIKFHDWIGDGWAILFSHPKNFTPVCTTELGTMAGLEGEFKKRNVKIIGISVDPVASHDKWQADIKTATGHSVHYPLIGDKDLKVAKLYDMLPAGAGETSEGRTPADNATVRSVYVIGPDKKIKLVLTYPMTTGRNFDEILRAVDSMQLTAKHQVATPANWKQGEDVIITAAVSNEDAIKRFGAYETILPYLRKTKQPSA

(19) United States
(12) Patent Application Publication
(10) Pub. No.: US 2025/0051823 A1
(43) Pub. Date: Feb. 13, 2025

(54) PRODUCTS AND METHODS FOR HETEROLOGOUS EXPRESSION OF PROTEINS IN A HOST CELL

(71) Applicant: ABSCI CORPORATION, Vancouver, WA (US)

(72) Inventors: Miles Gander, Vancouver, WA (US); Amy E. Doerner, Vancouver, WA (US); Carla Risso, Vancouver, WA (US); Gregory Hannum, Vancouver, WA (US); Ariel Schwartz, Vancouver, WA (US)

(21) Appl. No.: 18/723,092

(22) PCT Filed: Dec. 13, 2022
(86) PCT No.: PCT/US2022/081429

§ 371 Date: Jun. 21, 2024

Related U.S. Application Data
(60) Provisional application No. 63/293,285, filed on Dec. 23, 2021.

(51) Int. Cl. C12P 21/02; C12N 9/08; C12N 15/62; C12N 15/70

(52) U.S. Cl. CPC C12P 21/02; C12N 9/0065; C12N 15/625; C12N 15/70; C12Y 111/01

(57) ABSTRACT

The disclosure provides products and methods for the heterologous expression of proteins in host cells. The provided expression constructs, bacterial cells and methods produce a PnlP-1 accessory protein that increases the yield of a heterologous protein of interest.

### CROSS-REFERENCE TO RELATED APPLICATION

This application claims priority to U.S. Provisional Application No. 63/293,285, filed Dec. 23, 2021, which is incorporated by reference herein in its entirety.

### FIELD

The disclosure provides products and methods for the heterologous expression of proteins in host cells. The provided expression constructs, host cells and methods produce a PnlP-1 accessory protein that increases the yield of a heterologous protein of interest.

### INCORPORATION BY REFERENCE OF THE SEQUENCE LISTING

This application contains, as a separate part of disclosure, a Sequence Listing in computer-readable form (Filename: 57385_SeqListing.XML; 3,457 bytes; Created: Dec. 7, 2022) which is incorporated by reference herein in its entirety.

### BACKGROUND

One of the core challenges of heterologous protein expression in host cells such as E. coli is the formation of misfolded protein structure or insoluble protein product. In the natural E. coli system, molecular chaperone proteins mediate the proper folding of cellular proteins. Heterologous proteins expressed in E. coli are commonly unable to be properly folded and solubilized by native E. coli chaperones. It has been shown that expressing non-native molecular chaperones from organisms can help mediate proper folding of heterologous proteins. Thus, identification of new non-native chaperones that facilitate folding is an ongoing area of development.

There remains a need in the art for improved host cells and methods for heterologous protein expression.

### SUMMARY

The disclosure provides products and methods for manufacturing heterologous proteins in host cells.

A protein of the gram negative bacterium Mesorhizobium sp. Root172 is shown herein to increase the titer of heterologous proteins of interest in host cells, such as bacterial host cells. The protein is referred to herein as “PnlP-1” accessory protein. The protein was previously referred to at www.uniprot.org/uniprot/A0A0Q8KHV0 as a putative alkyl hydroperoxide reductase C. The amino acid sequence of the PnlP-1 accessory protein is set out in SEQ ID NO: 2.

The disclosure provides an expression construct comprising: a) a polynucleotide encoding an accessory protein of SEQ ID NO: 2, b) a polynucleotide encoding an accessory protein comprising an amino acid sequence at least 90% identical to SEQ ID NO: 2, or c) a polynucleotide encoding an accessory protein of SEQ ID NO: 2 or encoding an accessory protein comprising an amino acid sequence at least 90% identical to SEQ ID NO: 2, and a polynucleotide encoding a protein of interest. The accessory protein can be a Mesorhizobium sp. Root172 protein. The accessory protein can be a fusion protein comprising a heterologous signal peptide.

The polynucleotide encoding the accessory protein in an expression construct provided herein can comprise SEQ ID NO: 1. The polynucleotide encoding the accessory protein can be operably linked to a heterologous promoter. The heterologous promoter can be an inducible promoter or a constitutive promoter. The expression construct can be an extrachromosomal construct. The expression construct can further comprise one or more of: a heterologous promoter operably linked to a polynucleotide encoding a protein of interest; a bacterial origin of replication; and a ribosome binding site.

The protein of interest encoded by an expression construct provided herein can be an antibody product, a T cell receptor, a chimeric antigen receptor, an enzyme, or a fragment of any thereof. The protein of interest can contain one or more di-sulfide bonds.

The disclosure provides host cells comprising: a) an expression construct provided herein, or b) an expression construct provided herein which does not include a polynucleotide encoding a protein of interest and a second expression construct comprising a polynucleotide encoding a protein of interest. The host cell can be a prokaryotic cell. The prokaryotic host cell can be an E. coli cell. The host cell can be a eukaryotic cell. The eukaryotic host cell can be a yeast cell, insect cell or mammalian cell.

Host cells provided herein can comprise one or more of: a) an alteration of gene function of at least one gene encoding a transporter protein for an inducer of at least one inducible promoter; b) a reduced level of gene function of at least one gene encoding a protein that metabolizes an inducer of at least one inducible promoter; c) a reduced level of gene function of at least one gene encoding a protein involved in biosynthesis of an inducer of at least one inducible promoter; d) an altered gene function of a gene that affects the reduction/oxidation environment of the host cell cytoplasm; e) a reduced level of gene function of a gene that encodes a reductase; f) at least one expression construct encoding at least one disulfide bond isomerase protein; g) at least one polynucleotide encoding a form of DsbC lacking a signal peptide; and h) at least one polynucleotide encoding Ervlp.

The disclosure provides methods for producing a protein of interest comprising incubating a host cell provided herein under conditions that allow expression of the protein of interest. The methods can further comprise purifying the protein of interest.

The disclosure also provides methods of increasing the titer of properly folded proteins of interest comprising incubating a host cell provided herein under conditions that allow expression of the protein of interest.

### DETAILED DESCRIPTION

As indicated above, the disclosure provides products and methods for manufacturing heterologous protein products in a host cell.

“Host cells” herein are cells used in bioprocessing to manufacture heterologous protein products. Such host cells can be prokaryotic cells (for example, bacterial cells) or eukaryotic cells [for example, yeast cells or Chinese hamster ovary (CHO) cells]. E. coli host cells are exemplified herein.

“Heterologous” expression and “heterologous” protein product as used herein with reference to a host cell, refer to a protein product that is not naturally expressed by the host cell. For example, a heterologous protein product can refer to an accessory protein not naturally expressed by the host cell (e.g., PnlP-1 accessory protein of SEQ ID NO: 2 expressed in a cell other than the gram negative bacterium Mesorhizobium sp. Root172), or to a protein of interest to be manufactured in the host cell that is not naturally expressed by the host cell. “Heterologous” used in other contexts, such as in reference to a heterologous expression control element (e.g., promoter) operably linked to a polynucleotide encoding an accessory protein herein, in the same way indicates that the two components do not occur together in nature in that configuration.

Proteins of interest include any protein manufactured by bioprocessing where the protein of interest is heterologous to the host cell. Proteins of interest include, but are not limited to, antibody products. Antibody products can be, for example, a whole antibody, a single-chain variable fragment, a Fv, a Fab, a Fab′, a F(ab′) 2, a diabody, a triabody, a tetrabody, a Fd, a dAb, a minibody, or a maxibody. The antibody product may be a bispecific or multispecific antibody product.

### Expression Constructs

Expression constructs provided herein encode a PnlP-1 accessory protein. The same expression construct encoding a PnlP-1 accessory protein, or a separate expression construct, can encode a heterologous protein product to be manufactured.

Expression constructs provided herein include, for example, the codon optimized polynucleotide of SEQ ID NO: 1 which encodes the PnlP-1 accessory protein of SEQ ID NO: 2.

PnIP-1 polynucleotide sequence
(SEQ ID NO: 1)
ATGTCCCTTCGCATTAACGATATCGCCCCGGACTTCACCGCTGAA
ACGACGCAGGGCGAAATTAAGTTTCATGATTGGATTGGGGACGGA
TGGGCTATTCTGTTTAGTCACCCAAAAAATTTCACGCCCGTATGC
ACCACTGAACTGGGTACGATGGCTGGGTTAGAAGGTGAATTTAAG
AAAAGAAATGTGAAAATTATTGGCATATCTGTAGACCCTGTTGCT
TCACATGACAAATGGCAGGCAGATATCAAGACAGCCACTGGACAC
TCAGTGCACTATCCACTTATCGGTGATAAAGACCTGAAGGTCGCC
AAACTGTATGATATGTTACCAGCCGGTGCAGGCGAAACGTCAGAA
GGTCGCACCCCAGCGGATAATGCTACGGTGCGTAGTGTGTATGTA
ATTGGTCCGGATAAAAAAATTAAACTGGTTCTGACCTACCCTATG
ACGACTGGCCGCAATTTTGATGAAATTTTAAGAGCTGTTGATTCG
ATGCAACTGACCGCTAAACACCAAGTTGCTACGCCGGCAAACTGG
AAACAAGGAGAAGATGTTATAATCACCGCAGCCGTCTCAAATGAA
GATGCAATAAAACGGTTTGGGGCGTATGAAACCATCCTTCCGTAC
CTGAGAAAAACTAAACAACCTAGCGCG

This sequence was codon optimized via the IDT algorithm for expression in E. coli.

PnIP-1 polypeptide sequence
(SEQ ID NO: 2)
MSLRINDIAPDFTAETTQGEIKFHDWIGDGWAILFSHPKNFTPVC
TTELGTMAGLEGEFKKRNVKIIGISVDPVASHDKWQADIKTATGH
SVHYPLIGDKDLKVAKLYDMLPAGAGETSEGRTPADNATVRSVYV
IGPDKKIKLVLTYPMTTGRNFDEILRAVDSMQLTAKHQVATPANW
KQGEDVIITAAVSNEDAIKRFGAYETILPYLRKTKQPSA

Also provided herein are expression constructs including polynucleotides that encode a PnlP-1 accessory protein activity, wherein the polynucleotides are at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. As one example, a polynucleotide can be codon optimized to include codons preferred by the host cell in which it is expressed. As another example, a polynucleotide can encode a PnlP-1 protein that includes amino acid substitutions, insertions, or deletions, while retaining PnlP-1 accessory protein activity.

Assays to measure accessory protein activity include, PhyTip-based column target heterologous protein expression level quantification (phynexus.com/products/proteins/antibody-binding-phytip-columns/), flow cytometry-based ACE ASSAY™ measuring bound probe to properly folded target protein material (WO2021/146626), and/or an ELISA-based method HiPr bind assay (WO2021/163349), which measures fluorescence signal in a plate-based format of probes binding to properly folded target protein. These methods measure the increase in target protein production in the presence of the accessory protein compared to the production level in its absence. The increase can be at least 1.5-fold, at least two-fold, at least three-fold, at least four-fold, at least five-fold, at least six-fold, at least seven-fold, at least eight-fold, at least nine-fold, at least ten-fold, at least twenty-fold, at least fifty-fold, at least one hundred-fold, or greater.

Also provided herein are expression constructs comprising a polynucleotide that encodes a PnlP-1 protein with accessory protein activity, wherein the polynucleotide hybridizes under stringent conditions to SEQ ID NO: 1, or the complement thereof.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

Other expression constructs provided herein encode, for example, a PnlP-1 protein with accessory protein activity, wherein the protein comprises an amino acid sequence that is at least: 90%, 91%, 92%, 93%, 94%, 95%, 96%, 97%, 98%, or 99% identical to the PnlP-1 accessory protein of SEQ ID NO: 2. As an example, an expression construct can encode a PnlP-1 protein with amino acid substitutions in comparison to SEQ ID NO: 2 but the protein retains PnlP-1 accessory protein activity. As another example, an expression construct can encode a PnlP-1 protein with amino acid insertions or deletions in comparison to SEQ ID NO: 2 but the protein retains PnlP-1 accessory protein activity.

The terms “identical” and “identity” as used herein refer to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides are standard in the art. Methods can include those described in Computational Molecular Biology, (Lesk, Ed.), 1988, New York: Oxford University Press; Biocomputing Informatics and Genome Projects, (Smith, Ed.), 1993, New York: Academic Press; Computer Analysis of Sequence Data, Part I, (Griffin and Griffin, Eds.), 1994, New Jersey: Humana Press; Sequence Analysis in Molecular Biology, (von Heinje), 1987, New York: Academic Press; Sequence Analysis Primer, (Gribskov and Devereux, Eds.), 1991, New York: M. Stockton Press; and Carillo et al., SIAM J. Applied Math., 48:1073 (1988).

In calculating percent identity, the sequences being compared are aligned in a way that gives the largest match between the sequences. An exemplary computer program used to determine percent identity is the GCG program package, which includes GAP (Devereux et al., Nucl Acid Res, 12:387 (1984); Genetics Computer Group, University of Wisconsin, Madison, Wisc.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3.times. the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix [e.g., Dayhoff et al., Atlas of Protein Sequence and Structure, 5:345-352 (1978) for the PAM 250 comparison matrix; Henikoff et al., Proc. Natl. Acad. Sci. USA, 89:10915-10919 (1992) for the BLOSUM 62 comparison matrix] can also be used by the algorithm.

Recommended parameters for determining percent identity for polypeptides or nucleotide sequences using the GAP program are the following: Algorithm: Needleman et al., J. Mol. Biol., 48:443-453 (1970); Comparison matrix: BLOSUM 62 from Henikoff et al., 1992, supra; Gap Penalty: 12 (but with no penalty for end gaps); Gap Length Penalty: 4; Threshold of Similarity: 0.

Certain alignment schemes for aligning two amino acid sequences can result in matching of only a short region of the two sequences, and this small aligned region can have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

Other exemplary programs that compare and align pairs of sequences include, but are not limited to, ALIGN (Myers and Miller, Comput Appl Biosci, 19, 4 (1): 11-17 (1988), FASTA (Pearson and Lipman, Proc Natl Acad Sci USA, 85 (8): 2444-2448 (1988); Pearson, Methods Enzymol, 183:63-98 (1990) and gapped BLAST (Altschul et al., Nucleic Acids Res, 25 (17): 3389-40 (1997), BLASTP, BLASTN, or GCG (Devereux et al., Nucleic Acids Res, 12 (1 Pt 1): 387-95 (1984).

Expression constructs provided herein can encode a fragment of the PnlP-1 accessory protein (SEQ ID NO: 2), wherein the fragment retains PnlP-1 accessory protein activity.

“Expression constructs” herein are polynucleotides designed for the expression of one or more gene products of interest, and are not naturally occurring polynucleotide molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more gene products. One or more expression constructs can be integrated into a host cell chromosome or be maintained on an extrachromosomal polynucleotide such as a plasmid or artificial chromosome. The following are descriptions of various types of polynucleotide sequences that can be used in expression constructs for the expression or coexpression of gene products, including accessory proteins and protein products of interest as described herein.

Origins of replication. Expression constructs must comprise an origin of replication, also called a replicon, in order to be maintained within the host cell as independently replicating polynucleotides. Different replicons that use the same mechanism for replication cannot always be maintained together in a single host cell through repeated cell divisions. In those cases, plasmids can be categorized into incompatibility groups depending on the origin of replication that they contain, as shown in Table 2 of WO2016/205570. Origins of replication can be selected for use in expression constructs on the basis of incompatibility group, copy number, and/or host range, among other criteria. As described above, if two or more different expression constructs are to be used in the same host cell for the coexpression of multiple gene products, it is best if the different expression constructs contain origins of replication from different incompatibility groups: a pMBI replicon in one expression construct and a pl5A replicon in another, for example. The average number of copies of an expression construct in the cell, relative to the number of host chromosome molecules, is determined by the origin of replication contained in that expression construct. Copy number can range from a few copies per cell to several hundred (Table 2 of WO2016/205570). Different expression constructs can be used which comprise inducible promoters that are activated by the same inducer, but which have different origins of replication. By selecting origins of replication that maintain each different expression construct at a certain approximate copy number in the cell, it is possible to adjust the levels of overall production of a gene product expressed from one expression construct, relative to another gene product expressed from a different expression construct. As an example, to coexpress subunits A and B of a multimeric protein, an expression construct is created which comprises the colEl replicon, the am promoter, and a coding sequence for subunit A expressed from the am promoter: ‘colEl-Para-A.

Another expression construct is created comprising the pl 5A replicon, the am promoter, and a coding sequence for subunit B: ‘pl5A-Para-B’. These two expression constructs can be maintained together in the same host cells, and expression of both subunits A and B is induced by the addition of one inducer, arabinose, to the growth medium. If the expression level of subunit A needed to be significantly increased relative to the expression level of subunit B, in order to bring the stoichiometric ratio of the expressed amounts of the two subunits closer to a desired ratio, for example, a new expression construct for subunit A could be created, having a modified pMB 1 replicon as is found in the origin of replication of the pUC9 plasmid (‘pUC9ori’): pUC9ori-Para-A. Expressing subunit A from a high-copy-number expression construct such as pUC9ori-Para-A should increase the amount of subunit A produced relative to expression of subunit B from pl5A-Para-B. In a similar fashion, use of an origin of replication that maintains expression constructs at a lower copy number, such as pSOOI (WO2016/205570), could reduce the overall level of a gene product expressed from that construct. Selection of an origin of replication can also determine which host cells can maintain an expression construct comprising that replicon. For example, expression constructs comprising the colEl origin of replication have a relatively narrow range of available hosts, species within the Enterobacteriaceae family, while expression constructs comprising the RK2 replicon can be maintained in E. coli, Pseudomonas aeruginosa, Pseudomonas putida, Azotobacter vinelandii, and Alcaligenes eutrophus, and if an expression construct comprises the RK2 replicon and some regulator genes from the RK2 plasmid, it can be maintained in host cells as diverse as Sinorhizobium meliloti, Agrobacterium tumefaciens, Caulobacter crescentus, Acinetobacter calcoaceticus, and Rhodobacter sphaeroides (Kiies and Stahl, Microbiol Rev 1989 December; 53 (4): 491-516).

Similar considerations can be employed to create expression constructs for inducible expression or coexpression in eukaryotic cells. For example, the 2-micron circle plasmid of Saccharomyces cerevisiae is compatible with plasmids from other yeast strains, such as pSRI (ATCC Deposit Nos. 48233 and 66069; Araki et al., J Mol Biol 1985 Mar. 20; 182 (2): 191-203) and pKDI (ATCC Deposit No. 37519; Chen et al, Nucleic Acids Res 1986 Jun. 11; 14 (11): 4471-4481).

Selectable markers. Expression constructs usually comprise a selection gene, also termed a selectable marker, which encodes a protein necessary for the survival or growth of host cells in a selective culture medium. Host cells not containing the expression construct comprising the selection gene will not survive in the culture medium. Typical selection genes encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmIR or CmR), kanamycin (KanR), spectinomycin (SpcR), streptomycin (StrR), and tetracycline (TetR). Many of the plasmids in Table 2 of WO2016/205570 comprise selectable markers, such as pBR322 (AmpR, TetR); pMOB45 (CmR, TetR); pACYCIW (AmpR, KanR); and pGBMI (SpcR, StrR). The native promoter region for a selection gene is usually included, along with the coding sequence for its gene product, as part of a selectable marker portion of an expression construct. Alternatively, the coding sequence for the selection gene can be expressed from a constitutive promoter.

In various aspects, suitable selectable markers include, but are not limited to, neomycin phosphotransferase (npt II), hygromycin phosphotransferase (hpt), dihydrofolate reductase (dhfr), zeocin, phleomycin, bleomycin resistance gene (ble), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2), orotidine-5′-phosphate (OMP) decarboxylase (ura3), and orthologs of any of the foregoing.

Inducible promoter. As described herein, there are several different inducible promoters that can be included in expression constructs as part of the inducible coexpression systems of the disclosure. Inducible promoters share at least 80% polynucleotide sequence identity, at least 90% identity, or at least 95% identity to at least 30 at least 40, or at least 50 contiguous bases of a promoter polynucleotide sequence as defined in Table 1 of WO2016/205570 by reference to the E. coli K-12 substrain MG1655 genomic sequence, where percent polynucleotide sequence identity is determined using the methods of Example 11 of WO2016/205570. Under ‘standard’ inducing conditions (see Example 5 of WO2016/205570), preferred inducible promoters have at least 75%, at least 100% or at least 110% of the strength of the corresponding ‘wild-type’ inducible promoter of E. coli K-12 substrain MG1655, as determined using the quantitative PCR method of De Mey et al. (Example 6 of WO2016/205570). Within the expression construct, an inducible promoter is placed 5′ to (or “upstream of”) the coding sequence for the gene product that is to be inducibly expressed, so that the presence of the inducible promoter will direct transcription of the gene product coding sequence in a 5′ to 3′ direction relative to the coding strand of the polynucleotide encoding the gene product.

The following is a description of exemplary inducible promoters that can be used in expression constructs for expression or coexpression of gene products, along with some of the genetic modifications that can be made to host cells that contain such expression constructs. Examples of these inducible promoters and related genes are, unless otherwise specified, from Escherichia coli (E. coli) strain MG1655 (American Type Culture Collection deposit ATCC 700926), which is a substrain of E. coli K-12 (American Type Culture Collection deposit ATCC 10798). Table 1 of WO/2016/205570 lists the genomic locations, in E. coli MG1655, of the nucleotide sequences for these examples of inducible promoters and related genes. Nucleotide and other genetic sequences, referenced by genomic location as in Table 1 of WO/2016/205570, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose—araBAD, araC, arciE, and araFGH—but the terms ‘arabinose promoter’ and ‘ara promoter’ are typically used to designate the araBAD promoter. Several additional terms have been used to indicate the E. coli araBAD promoter, such as Para, ParaB, ParaBAD, and PBAD—The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from PBAD, but in the presence of arabinose, the AraC protein, which alters its conformation upon binding arabinose, becomes a positive regulatory element that allows transcription from PBAD—The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY (AIWC) and the LacY (AIWV) proteins, having a cysteine or a valine amino acid instead of alanine at position 177, respectively (Morgan-Kiss et al., Proc Natl Acad Sci USA 2002 May 28; 99 (11): 7373-7377). In order to achieve homogenous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY (A177C) or LacY (A177V), will not lead to the ‘all or none’ induction phenomenon when the arabinose-inducible promoter is induced by the presence of arabinose. Because the LacY (A177C) protein appears to be more effective in transporting arabinose into the cell, use of polynucleotides encoding the LacY (A177C) protein is preferred to the use of polynucleotides encoding the LacY (A177V) protein.

Propionate promoter. The ‘propionate promoter’ or ‘prp promoter’ is the promoter for the E. coli prpBCDE operon, and is also called PPB. Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription from the prp promoter when the PrpR protein binds 2-methylcitrate (‘2-MC’). Propionate (also called propanoate) is the ion, CH3CH2COO—, of propionic acid (or ‘propanoic acid’), and is the smallest of the ‘fatty’ acids having the general formula H(CH2),COOH that shares certain properties of this class of molecules: producing an oily layer when salted out of water and having a soapy potassium salt. Commercially available propionate is generally sold as a monovalent cation salt of propionic acid, such as sodium propionate (CH3CH2COONa), or as a divalent cation salt, such as calcium propionate (Ca(CH3CH2COO)2). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolism of 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-yg/DGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfl, introduced into E. coli BL21 (DE3) to create strain JSB (Lee and Keasling, “A propionate-inducible expression system for enteric bacteria”, Appl Environ Microbiol 2005 November; 71 (11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfl also apparently affects ygfl, which encodes a putative LysR-family transcriptional regulator of unknown function. The genes sbm-yg/DGH are transcribed as one operon, and ygfl is transcribed from the opposite strand. The 3′ ends of the ygfti and ygfl coding sequences overlap by a few base pairs, so a deletion that takes out all of the sbm-yg/DGH operon apparently takes out ygfl coding function as well. Eliminating or reducing the function of a subset of the sbm-ygfDGH gene products, such as YgfG (also called ScpB, methylmalonyl-CoA decarboxylase), or deleting the majority of the sbm-yg/DGH (or scpA-argK-scpBC) operon while leaving enough of the 3′ end of the ygfli (or scpC) gene so that the expression of ygfl is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter’ or ‘rha promoter’, or PrhaSR, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al, J Bacteriol 2010 January; 192 (1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxy acetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins RmIA, RmIB, RmIC, and RmID (also called RfbA, RfbB, RfbC, and RfbD, respectively) encoded by the rmIBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the RmIA, RmIB, RmIC, and

RmID proteins. L-rhamnose is transported into the cell by RhaT, the rhamnose permease or L-rhamnose: proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or PxyiA, means the promoter for the E. coli xylAB operon. The xylose promoter region is similar in organization to other inducible promoters in that the xylAB operon and the xylFGHR operon are both expressed from adjacent xylose-inducible promoters in opposite directions on the E. coli chromosome (Song and Park, J Bacteriol. 1997 November; 179 (22): 7025-7032). The transcriptional regulator of both the PxyiA and PxyiF promoters is XyIR, which activates expression of these promoters in the presence of xylose. The xyIR gene is expressed either as part of the xylFGHR operon or from its own weak promoter, which is not inducible by xylose, located between the xyIH and xyIR protein-coding sequences. D-xylose is catabolized by XyIA (D-xylose isomerase), which converts D-xylose to D-xylulose, which is then phosphorylated by XyIB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XyIA, or optionally of both XylA and XylB. The xylFGHR operon encodes XyIF, XyIG, and XyIH, the subunits of an ABC super-family high-affinity D-xylose transporter. The xyIE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xylFGHR operon could be altered so that the xyIFGH coding sequences are deleted, leaving XyIR as the only active protein expressed from the xylose-inducible PxyiF promoter, and with the xyIE coding sequence expressed from a constitutive promoter rather than its native promoter. As another example, the xyIR coding sequence is expressed from the PxyiA or the promoter in an expression construct, while either the xylFGHR operon is deleted and xyIE is constitutively expressed, or alternatively an xyIFGH operon (lacking the xyIR coding sequence since that is present in an expression construct) is expressed from a constitutive promoter and the xyIE coding sequence is deleted or altered so that it does not produce an active protein.

Lactose promoter. The term ‘lactose promoter’ refers to the lactose-inducible promoter for the lacZYA operon, a promoter which is also called lacZpl; this lactose promoter is located at ca. 365603-365568 (minus strand, with the NA polymerase binding (‘−35’) site at ca. 365603-365598, the Pribnow box (‘−10’) at 365579-365573, and a transcription initiation site at 365567) in the genomic sequence of the E. coli K-12 substrain MG1655 (NCBI Reference Sequence NC 000913.2, 11 Jan. 2012). Inducible coexpression systems of the disclosure can comprise a lactose-inducible promoter such as the lacZYA promoter. Inducible coexpression systems of the disclosure can comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘−10’) at 401695-401701 (Kikuchi et al., Nucleic Acids Res 1981 Nov. 11; 9 (21): 5671-5678)) in the genomic sequence of the E. coli K-12 substrain MG1655 (NCBI Reference Sequence NC 000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417,142-417,831 and the PhoR coding sequence at 417,889-419,184) in the genomic sequence of the E. coli K-12 substrain MG1655 (NCBI Reference Sequence NC 000913.3, 16 Dec. 2014). The phoA promoter differs from the inducible promoters described above in that it is induced by the lack of a substance-intracellular phosphate-rather than by the addition of an inducer. For this reason the phoA promoter is generally used to direct transcription of gene products that are to be produced at a stage when the host cells are depleted for phosphate, such as the later stages of fermentation. Inducible coexpression systems of the disclosure can comprise a phoA promoter. Inducible coexpression systems of the disclosure comprise one or more inducible promoters that are not phoA promoters.

Expression from constitutive promoters. Expression constructs of the disclosure can also comprise coding sequences that are expressed from constitutive promoters. Unlike inducible promoters, constitutive promoters initiate continual gene product production under most growth conditions. One example of a constitutive promoter is that of the Tn3 bla gene, which encodes beta-lactamase and is responsible for the ampicillin-resistance (AmpR) phenotype conferred on the host cell by many plasmids, including pBR322 (ATCC 31344), pACYCIW (ATCC 37031), and pBAD24 (ATCC 87399). Another constitutive promoter that can be used in expression constructs is the promoter for the E. coli lipoprotein gene, Ipp, which is located at positions 1755731-1755406 (plus strand) in E. coli K-12 substrain MG1655 (Inouye and Inouye, Nucleic Acids Res 1985 May 10; 13 (9): 3101-3110). A further example of a constitutive promoter that has been used for heterologous gene expression in E. coli is the trpLEDCBA promoter, located at positions 1321169-1321133 (minus strand) in E. coli K-12 substrain MG1655 (Windass et al., Nucleic Acids Res 1982 Nov. 11; 10 (21): 6639-6657). Constitutive promoters can be used in expression constructs for the expression of selectable markers, as described herein, and also for the constitutive expression of other gene products useful for the coexpression of the desired product. For example, transcriptional regulators of the inducible promoters, such as AraC, PrpR, RhaR, and XyIR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct. Gene products useful for the production of coexpressed gene products, and the resulting desired product, also include accessory protein proteins, cofactor transporters, etc.

Ribosome binding site. For polypeptide gene products, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. The region of the expression construct that corresponds to the 5′ UT can comprise a polynucleotide sequence similar to the consensus ribosome binding site (RBS, also called the Shine-Dalgarno sequence) that is found in the species of the host cell. In prokaryotes (archaea and bacteria), the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In expression constructs, the RBS sequence is at least 55% identical to the AGGAGGU consensus sequence, at least 70% identical, or at least 85% identical, and is separated from the initiation codon by 5 to 10 intervening nucleotides, by 6 to 9 intervening nucleotides, or by 6 or 7 intervening nucleotides. The ability of a given RBS to produce a desirable translation initiation rate can be calculated at the website salis.psu.edu/software/RBSLibraryCalculatorSearchMode, using the RBS Calculator; the same tool can be used to optimize a synthetic RBS for a translation rate across a 100,000+ fold range (Salis, Methods Enzymol 2011; 498:19-42).

Multiple cloning site. A multiple cloning site (MCS), also called a polylinker, is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS. Examples of MCS sequences are those in the pBAD series of expression vectors, including pBAD18, pBAD18-Cm, pBAD18-Kan, pBAD24, pBAD28, pBAD30, and pBAD33 (Guzman et al., J Bacteriol 1995 July; 177 (14): 4121-4130); or those in the pPRO series of expression vectors derived from the pBAD vectors, such as pPR018, pPR018-Cm, pPR018-Kan, pPR024, pPRO30, and pPR033 (U.S. Pat. No. 8,178,338 B2; May 15, 2012; Keasling, Jay). A multiple cloning site can be used in the creation of an expression construct: by placing a multiple cloning site 3′ to (or downstream of) a promoter sequence, the MCS can be used to insert the coding sequence for a gene product to be expressed or coexpressed into the construct, in the proper location relative to the promoter so that transcription of the coding sequence will occur. Depending on which restriction enzymes are used to cut within the MCS, there may be some part of the MCS sequence remaining within the expression construct after the coding sequence or other polynucleotide sequence is inserted into the expression construct. Any remaining MCS sequence can be upstream or, or downstream of, or on both sides of the inserted sequence. A ribosome binding site can be placed upstream of the MCS, immediately adjacent to or separated from the MCS by only a few nucleotides, in which case the RBS would be upstream of any coding sequence inserted into the MCS. Another alternative is to include a ribosome binding site within the MCS, in which case the choice of restriction enzymes used to cut within the MCS will determine whether the RBS is retained, and in what relation to, the inserted sequences. A further alternative is to include a RBS within the polynucleotide sequence that is to be inserted into the expression construct at the MCS, in the proper relation to any coding sequences to stimulate initiation of translation from the transcribed messenger RNA.

Signal Peptides. Polypeptide gene products expressed or coexpressed by the methods of the disclosure can contain signal peptides or lack them, depending on whether it is desirable for such gene products to be exported from the host cell cytoplasm into the periplasm, or to be retained in the cytoplasm, respectively. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides can be characterized functionally by the ability to direct transport of a polypeptide, either co-translationally or post-translationally, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm, using a method such as described in Example 12 of WO2016/205570.

### Host Cells

Host cells provided herein comprise one or more expression constructs described herein.

Prokaryotic host cells. Host cells are provided comprising expression constructs designed for expressing heterologous gene products (such as accessory proteins described herein) in host cells including, but not limited to, prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Such host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

As described in WO2017/106583, incorporated by reference in its entirety herein, producing gene products such as therapeutic proteins at commercial scale and in soluble form is addressed, for example, by providing suitable host cells capable of growth at high cell density in fermentation culture, and which can produce soluble gene products in the oxidizing host cell cytoplasm through highly controlled inducible gene expression. Host cells of the present disclosure with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Specific examples of such genetic alterations are provided herein. Optionally, host cells can also be genetically modified to express accessory proteins and/or cofactors that assist in the production of the desired gene product(s), and/or to glycosylate polypeptide gene products. (2) The host cells comprise one or more expression constructs designed for the expression of one or more gene products of interest. At least one expression construct can comprise an inducible promoter and a polynucleotide encoding a gene product to be expressed from the inducible promoter. (3) The host cells contain additional genetic modifications designed to improve certain aspects of gene product expression from the expression construct(s). The host cells can (A) have an alteration of gene function of at least one gene encoding a transporter protein for an inducer of at least one inducible promoter, and as another example, wherein the gene encoding the transporter protein is selected from the group consisting of araE, araE, araG, araH, rhaT, xyIF, xyIG, and xyIH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is araA, araB, araD, prpB, prpD, rhaA, rhaB, rhaD, xylA, or xylB; and/or (C) have a reduced level of gene function of at least one gene encoding a protein involved in biosynthesis of an inducer of at least one inducible promoter, which gene is further scpA/sbm, argK/ygfD, scpB/ygfG, scpC/ygfH, rmIA, rmIB, rmIC, or rmID.

Host Cells with Oxidizing Cytoplasm. Host cells are provided that allow for the efficient and cost-effective expression of gene products, including components of multimeric products. Host cells can include, in addition to isolated cells in culture, cells that are part of a multicellular organism, or cells grown within a different organism or system of organisms. In certain embodiments of the disclosure, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli (B strain) EB0001 cells (also called E. coli ASE (DGH) cells), or are E. coli (B strain) EB0002 cells. In growth experiments with E. coli host cells having oxidizing cytoplasm, specifically the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H), these E. coli B strains with oxidizing cytoplasm are able to grow to much higher cell densities than the most closely corresponding E. coli K strain (WO2017/106583).

Alterations to host cell gene functions. Certain alterations can be made to the gene functions of host cells comprising inducible expression constructs, to promote efficient and homogeneous induction of the host cell population by an inducer. The combination of expression constructs, host cell genotype, and induction conditions can result in at least 75%, at least 85% or at least 95% of the cells in the culture expressing gene product from each induced promoter, as measured by the method of Khlebnikov et al. described in Example 9 of WO2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the gene protein-coding sequence in its entirety, or deleting a large enough portion of the gene, inserting sequence into the gene, or otherwise altering the gene sequence so that a reduced level of functional gene product is made from that gene. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of accessory proteins, it is also possible to alter the reduction-oxidation environment of the host cell.

Host cell reduction-oxidation environment. In bacterial cells such as E. coli, proteins that need disulfide bonds are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 2011 May 14; 10:32). It is also possible to express cytoplasmic forms of these Dsb proteins, such as a cytoplasmic version of DsbA and/or of DsbC (‘cDsbA or ‘cDsbC’), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in heterologous proteins produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (such as ahpC* and ahpCA, Lobstein et al., Microb Cell Fact 2012 May 8; 11:56; doi: 10.1186/1475-2859-11-56) in the gene ahpC, which encodes AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dupl62-169 (Faulkner et al., Proc Natl Acad Sci USA 2008 May 6; 105 (18): 6735-6740, Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase I) catalase-peroxidase encoded by E. coli katG and HPII (hydroperoxidase II) catalase-peroxidase encoded by E. coli katE, which disproportionate peroxide into water and 02 (Farr and Kogoma, Microbiol Rev. 1991 December; 55 (4): 561-585; Review). Increasing levels of KatG and/or KatE protein in host cells through induced coexpression or through elevated levels of constitutive expression is contemplated herein.

Another alteration that can be made to host cells is to express the sulfhydryl oxidase Ervlp from the inner membrane space of yeast mitochondria in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 2011 Jan. 7; 10:1).

Host cells comprising expression constructs can also express cDsbA and/or cDsbC and/or Ervlp; are deficient in trxB gene function; are also deficient in the gene function of either gor, gshB, or gshA; optionally have increased levels of katG and/or katE gene function; and express an appropriate mutant form of AhpC so that the host cells can be grown in the absence of DTT.

Chaperone proteins. Gene products of interest can be coexpressed with other gene products, such as chaperone proteins, that are beneficial to the production of the desired gene product. Chaperone proteins are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperone proteins can be expressed from an inducible promoter or a constitutive promoter within an expression construct, or can be expressed from the host cell chromosome; expression of chaperone protein protein(s) in the host cell is at a sufficiently high level to produce coexpressed gene products that are properly folded and/or assembled into the desired product. Examples of chaperone proteins present in E. coli host cells are the folding factors Dnak/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. Dnak/DnaJ/GrpE, GroEL/GroES, and ClpB can function synergistically in assisting protein folding and therefore expression of these chaperone proteins in combinations has been shown to be beneficial for protein expression (Makino et al., Microb Cell Fact 2011 May 14; 10:32). When expressing eukaryotic proteins in prokaryotic host cells, a eukaryotic chaperone protein, such as protein disulfide isomerase (PDI) from the same or a related eukaryotic species, can be coexpressed or inducibly coexpressed with the desired gene product.

One chaperone that can be expressed in host cells is a protein disulfide isomerase from Humicola insolens, a soil hyphomycete (soft-rot fungus). An amino acid sequence of Humicola insolens PDI is shown as SEQ ID NO: 1 of WO2017/106583; it lacks the signal peptide of the native protein so that it remains in the host cell cytoplasm. The nucleotide sequence encoding PDI was optimized for expression in E. coli; the expression construct for PDI is shown as SEQ ID NO: 2 of WO2017/106583. SEQ ID NO: 2 of WO2017/106583 contains a GCTAGC NheI restriction site at its 5′ end, an AGGAGG ribosome binding site at nucleotides 7 through 12, the PDI coding sequence at nucleotides 21 through 1478, and a GTCGAC Sail restriction site at its 3′ end. The nucleotide sequence of SEQ ID NO: 2 of WO2017/106583 was designed to be inserted immediately downstream of a promoter, such as an inducible promoter. The NheI and Sail restriction sites in SEQ ID NO: 2 of WO2017/106583 can be used to insert it into a vector multiple cloning site, such as that of the pSOL expression vector (SEQ ID NO: 3 of WO2017/106583), described in published US patent application US2015353940A1, which is incorporated by reference in its entirety herein. Other PDI polypeptides can also be expressed in host cells, including PDI polypeptides from a variety of species (Saccharomyces cerevisiae (UniProtKB PI 7967), Homo sapiens (UniProtKB P07237), Mus musculus (UniProtKB P09103), Caenorhabditis elegans (UniProtKB Q 17770 and Q 17967), Arabdopsis thaliana (UniProtKB 048773, Q9XI01, Q9S G3, Q9LJU2, Q9MAU6, Q94F09, and Q9T042), Aspergillus niger (UniProtKB Q12730) and also modified forms of such PDI polypeptides. A PDI polypeptide expressed in host cells of the disclosure can share at least 70%, or 80%, or 90%, or 95% amino acid sequence identity across at least 50% (or at least 60%, or at least 70%, or at least 80%, or at least 90%) of the length of SEQ ID NO: I of WO2017/106583, where amino acid sequence identity is determined according to Example 10 of WO2017/106583.

Cellular transport of cofactors. When using the expression systems of the disclosure to produce enzymes that require cofactors for function, it is helpful to use a host cell capable of synthesizing the cofactor from available precursors, or taking it up from the environment. Common cofactors include ATP, coenzyme A, flavin adenine dinucleotide (FAD), NAD+/NADH, and heme. Polynucleotides encoding cofactor transport polypeptides and/or cofactor synthesizing polypeptides can be introduced into host cells, and such polypeptides can be constitutively expressed, or inducibly coexpressed with the gene products to be produced by methods of the disclosure.

Glycosylation of polypeptide gene products. Host cells can have alterations in their ability to glycosylate polypeptides. For example, eukaryotic host cells can have eliminated or reduced gene function in glycosyltransferase and/or oligo-saccharyltransferase genes, impairing the normal eukaryotic glycosylation of polypeptides to form glycoproteins. Prokaryotic host cells such as E. coli, which do not normally glycosylate polypeptides, can be altered to express a set of eukaryotic and prokaryotic genes that provide a glycosylation function (DeLisa et al., WO2009/089154A2, 2009 Jul. 16).

Available host cell strains with altered gene functions. To create preferred strains of host cells to be used in the expression systems and methods of the disclosure, it is useful to start with a strain that already comprises desired genetic alterations (See Table A of WO2017/106583, reproduced below).

Host Cell Strains

| Strain: | Genotype: | Source: |
|---|---|---|
| E. coli | F-mcrA Δ(mrr-hsdRMS-mcrBC) | Invitrogen Life Technologies |
| TOP10 | φ80lacZ ΔM15 lacX74 recAl araD139 | Catalog nos. C4040-10, |
| | Δ(ara-leu) 7697 galU galK rpsL (Str^{R}) | C4040-03, C4040-06, C4040- |
| | endA1 nupGλ- | 50, and C4040-52 |
| E. coli | Δ(ara-leu) 7697 ΔlacX74 ΔphoA Pvull phoR | Merck (EMD Millipore |
| Origami ™ 2 | araD 139 ahpC galE galK rpsL F′[lac⁺ lacl^{q} pro] | Chemicals) Catalog No. 71344 |
| | gor522::Tn10 trxB (Str^{R}, Tet^{R}) | |
| E. coli | fhuA2 [Ion] ompT ahpC gal λatt::pNEB3-r1- | New England Biolabs Catalog |
| SHuffle ® | cDsbC (Spec, lac1) ΔtrxB sulA11 R(mcr- | No. C3028H |
| Express | 73::miniTn10--Tet^{s})2 [dcm] R(zgb-210::Tn10-- | |
| | Tet^{s}) endAl Δgor A(mcrC-mrr)114::IS10 | |

### Assays for Accessory Protein Activity

Accessory protein activity can be measured, for example, by the following assays. For example, the accessory protein activity of a PnlP-1 variant or fragment can be demonstrated by one or more of the assays.

Exemplary ACE assay protocol (based on assays in WO2021/146626)
1. Fix sample cells with a formaldehyde based solution.
2. Prepare a permeablization buffer and treat cells.
3. Add biotin to the 1×PE+ 1 mM EDTA to a final concentration 0.1 mg/ml biotin.
4. Add biotin (stored at −80° C. or 4° C.) at a 100× dilution (e.g., 5000 μL or 5 mL of PE buffer would require 50 μL biotin).
5. Combine the primary (and secondary probes if dual probe), fluorescently labled in 1×PE+ 1 mM EDTA in 15 ml centrifuge tube (or 50 mL, if staining reagent volume exceeds 15 mL)
6. Incubate and rotate for at least 1 hour at 4° C. with foil wrapped around tube.
7. After 1 hr, add biotin to stain solution to a final concentration 0.1 mg/ml biotin
8. Add biotin (stored at −80 or 4° C.) at a 100× dilution. Eg, 5000 μL or 5 mL of PE buffer would require 50 μL biotin) Incubate again with rotation for at least 30 min with foil wrapped around tube.
9. Spin samples at 3300 g at 4° C. for 5 minutes
10. Aspirate the supernatant with the vacuum pump and the matrix tube attachment. Avoid touching the attachment to the sides of the tube
11. Slowly cascade 500 μL of 1×PBS+ 1 mM EDTA onto the side of the sample tubes without disturbing the pellet.
12. Add 250 μL of E2 Fixation Buffer to each tube.
13. After the 18 hr incubation, remove samples from rotator and spin in centrifuge at 3300 g at 4° C. for 3 minutes.
14. Carry out FACS on the stained cell samples, binning by fluorescence signal.

Exemplary HiPR Bind assay (based on assays in WO2021/163349)
1. Culture sample cells in proper induction conditions to facilitate target protein and accessory protein expression. Ie arabinose and or proprionate media. Grow sample cells in a 96 well plate.
2. Keep sample plates on ice to thaw. While those are thawing, gather and label the plates needed and prep assay solutions.
3. Dilute standard into Dilution Buffer 1 solution (0.1× Perkin Elmer Buffer, 1 mM EDTA, 1×PBS).
4. Prepare Assay solution I (ASI) and Assay solution II (ASII) in dark amber colored 50 mL conicals.
5. Predispense Dilution Buffer 2 into 384 well V-bottom Greiner Bio-One dilution plates, resuspend cell pellets
6. Predispense ASI into 384-well Proxiplates. Visually inspect that the silicone nozzles are fitted properly and that nothing looks loose or off kilter.
7. Once each plate is finished, seal it with a Perkin Elmer plate sealer.
8. Spin down plates at 500 g for 1 min.
9. Incubate the plates overnight at 4° C.
10. The next day, take the plates out of 4° C. storage. Allow to equilibrate to room temp for at least 1 hour.
11. Feed plates into plate feeder on the Enspire.
12. Scan on the Enspire using the “Alpha, FI-DNA_Ex480 Em 520-Alpha 384-SW”.
13. Record values for further analysis of alpha max slope.

### Other Terminology and Disclosure

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “can comprise” or “can be” indicates something envisaged by the inventors that is functional and available as part of the subject matter provided.

While the following examples describe specific embodiments, variations and modifications will occur to those skilled in the art. Accordingly, only such limitations as appear in the claims should be placed on the invention.

### EXAMPLES

The following experiments identified an accessory protein that significantly increased the titer of three different proteins expressed in heterologous host cells.

### Example 1

E. coli host cells expressing a Fab molecule (referred to herein as Fab 1) were transformed with two plasmid accessory protein libraries derived from the DENOVIUM ENGINE and were cultivated via an AMBR fermentation system (Sartorius). Using an ACE assay described in WO2021/146626, the resulting host cells were binned and sorted across the distribution of specific cell activity. The plasmid DNA from the cells of each of the activity bins was extracted and sequenced using shotgun sequencing (Illumina). Sequencing reads were computationally aligned to accessory protein coding sequences within the accessory protein library. Fractional read abundance was measured, and enrichment scores and p values were calculated based on the fractional abundance of each accessory protein across all of the activity bins.

For the Fab 1 molecule, the two libraries shared a handful of high enrichment score accessory proteins, one of which comprised the amino acid sequence of SEQ ID NO: 2, a sequence which has previously been inferred by homology to encode a peroxidase as mentioned above. Host cells strains expressing this protein were chosen for further evaluation of its activity as an accessory protein for enhancing production of Fab 1 in the host cells.

### Example 2

Based on the ACE enrichment analysis described above, eleven individual host cell strains containing plasmids with different combinations of accessory proteins that included the protein of SEQ ID NO: 2, were grown using the Ambr® fermentation system. Each strain was grown in duplicate. Each strain was assayed for Fab 1 titer using Antibody Binding Phy Tip Columns at different sampling timepoints during the fermentation run.

Three strains containing plasmids encoding the protein of SEQ ID NO: 2, named “PnlP-1” accessory protein herein, showed increased protein production over the positive control strain.

### Example 3

Using the same host cells, plasmid libraries, and methods as in Example 1, accessory protein enrichment scores were calculated via ACE for another Fab molecule (referred to as Fab 2 herein).

Comparing the enrichment scores for the Fab 1 and Fab 2 molecules, there were some accessory proteins that appeared in the lowest score set for both Fab 1 and Fab 2, and some accessory proteins that appeared in the highest score set for both Fab 1 and Fab 2. The host cell strains expressing PnlP-1 accessory protein again appeared in the highest set of scores, indicating its utility for increasing expression across different Fab molecules.

### Example 4

An ELISA-based method was used to assess titer of another heterologous protein, an Fc fusion molecule, produced in the same type of E. coli host cells. The method was applied to multiple accessory protein plasmid-containing E. coli strains that expressed the Fc fusion molecule (referred to herein as Fc fusion 1). The strains were grown in 96-well 1 ml deep well plates. Strains were grown and tested in triplicate. The cell culture was harvested and lysed as input for the ELISA test.

The host cell strain that had the highest ELISA signal for the Fc fusion 1 molecule expressed PnlP-1 accessory protein, indicating the utility of this accessory protein for increasing expression across different heterologous protein biologic classes.

All documents referred to in this application are hereby incorporated by reference in their entirety.

We claim:

1. An expression construct comprising:
a) a polynucleotide encoding an accessory protein of SEQ ID NO: 2,
b) a polynucleotide encoding an accessory protein comprising an amino acid sequence at least 90% identical to SEQ ID NO: 2, or
c) a polynucleotide encoding an accessory protein of SEQ ID NO: 2 or encoding an accessory protein comprising an amino acid sequence at least 90% identical to SEQ ID NO: 2, and a polynucleotide encoding a protein of interest.

2. The expression construct of claim 1 wherein the accessory protein is a Mesorhizobium sp. Root172 protein.

3. The expression construct of claim 1 or 2 wherein the accessory protein comprises SEQ ID NO: 2.

4. The expression construct of claim 1, 2 or 3 wherein the accessory protein is a fusion protein comprising a heterologous signal peptide.

5. The expression construct of claim 1, 2, 3, or 4 wherein the polynucleotide encoding the accessory protein comprises SEQ ID NO: 1.

6. The expression construct of claim 1, 2, 3, 4 or 5 wherein the polynucleotide encoding the accessory protein is operably linked to a heterologous promoter.

7. The expression construct of claim 6 wherein the heterologous promoter is an inducible promoter or a constitutive promoter.

8. The expression construct of claim 1, 2, 3, 4, 5, 6 or 7 wherein the expression construct is an extrachromosomal construct.

9. The expression construct of claim 1, 2, 3, 4, 5, 6, 7 or 8 further comprising one or more of a heterologous promoter, a bacterial origin of replication and a ribosome binding site.

10. The expression construct of claim 1, 2, 3, 4, 5, 6, 7, 8 or 9 wherein the protein of interest is an antibody product, a T cell receptor, a chimeric antigen receptor, an enzyme, or a fragment of any thereof.

11. The expression construct of claim 1, 2, 3, 4, 5, 6, 7, 8, 9 or 10 wherein the protein of interest contains one or more di-sulfide bonds.

12. A host cell comprising:
a) an expression construct according to any preceding claim, or
b) an expression construct according to a preceding claim which does not include a polynucleotide encoding a protein of interest and a second expression construct comprising a polynucleotide encoding a protein of interest.

13. The host cell of claim 12 wherein the host cell is a prokaryotic cell.

14. The host cell of claim 13 wherein the host cell is an E. coli cell.

15. The host cell of claim 14 wherein the host cell is a eukaryotic cell.

16. The host cell of claim 15 wherein the host cell is a yeast cell, insect cell or mammalian cell.

17. The host cell of claim 12 wherein the cell comprises one or more of:
a) an alteration of gene function of at least one gene encoding a transporter protein for an inducer of at least one inducible promoter;
b) a reduced level of gene function of at least one gene encoding a protein that metabolizes an inducer of at least one inducible promoter;
c) a reduced level of gene function of at least one gene encoding a protein involved in biosynthesis of an inducer of at least one inducible promoter;
d) an altered gene function of a gene that affects the reduction/oxidation environment of the host cell cytoplasm;
e) a reduced level of gene function of a gene that encodes a reductase;
f) at least one expression construct encoding at least one disulfide bond isomerase protein;
g) at least one polynucleotide encoding a form of DsbC lacking a signal peptide; and
h) at least one polynucleotide encoding Ervlp.

18. A method for producing a protein of interest comprising incubating a host cell according to claim 10, 11, 12, 13, 14, 15, 16 or 17 under conditions that allow expression of the protein of interest.

19. The method of claim 18 further comprising purifying the protein of interest.

20. A method of increasing the titer of properly folded proteins of interest comprising incubating a host cell according to claim 10, 11, 12, 13, 14, 15, 16 or 17 under conditions that allow expression of the protein of interest.
